(19) United States
(12) Patent Application Publication
(10) Pub. No.: US 2026/0294341 A1
(43) Pub. Date: Oct. 1, 2026

(54) HEARABLE DEVICE, INTEGRATED CIRCUIT, AND BIOLOGICAL SIGNAL MEASUREMENT SYSTEM

(71) Applicant: SONY GROUP CORPORATION, Tokyo (JP)

(72) Inventors: Mao KATSUHARA, Tokyo (JP); Takanori ISHIKAWA, Tokyo (JP); Yukinojo KITAKAMI, Tokyo (JP); Yota KOMORIYA, Tokyo (JP)

(21) Appl. No.: 19/476,254

(22) PCT Filed: Apr. 8, 2024
(86) PCT No.: PCT/JP2024/014279

§ 371 Date: Oct. 17, 2025

(30) Foreign Application Priority Data
Apr. 27, 2023 (JP) 2023-073646

(51) Int. Cl. A61B 5/00; A61B 5/0205; A61B 5/024; A61B 5/16; A61B 5/291; A61B 5/372; H04R 1/08; H04R 1/10

(52) U.S. Cl. CPC A61B 5/6817; A61B 5/0205; A61B 5/165; A61B 5/291; A61B 5/372; A61B 5/721; H04R 1/08; H04R 1/1016; A61B 5/02416; A61B 5/02438; A61B 2562/0219; H04R 2201/003; H04R 2410/05

(57) ABSTRACT

The present disclosure relates to a hearable device, an integrated circuit, and a biological signal measurement system that enable realization of acquisition of a cleaner biological signal.

A noise reduction unit executes noise reduction of a biological signal of a user measured by a biological sensor using an output signal from at least one of a microphone or an inertial sensor as a reference signal. The present disclosure can be applied to a hearable device such as a TWS earphone.

### TECHNICAL FIELD

The present disclosure relates to a hearable device, an integrated circuit, and a biological signal measurement system, and more particularly to a hearable device, an integrated circuit, and a biological signal measurement system that enable realization of acquisition of a cleaner biological signal.

### BACKGROUND ART

In recent years, various services using a biological signal measurement technology around the ear by an EEG sensor, a PPG sensor, or the like incorporated in a hearable device such as an earphone or a headphone have been proposed in many cases.

Non-Patent Document 1 proposes a technology for removing noise using a signal of a microphone for an intra-aural electroencephalogram.

### CITATION LIST

### Non-Patent Document

Non-Patent Document 1: V. Goverdovsky, W. V. Rosenberg, T. Nakamura, D. Looney, D. J. Sharp, C. Papavassiliou, M. J. Morrell, and D. P. Mandic, “Hearables: Multimodal physiological in-ear sensing” Scientific Reports, vol. 7, Article no. 6948, 2017

### SUMMARY OF THE INVENTION

### Problems to be Solved by the Invention

However, in a use condition actually assumed for a hearable device, there is a possibility that more noise is mixed in a body motion of a user and an external environment. In addition, as exemplified in the technology of Non-Patent Document 1, it is practically difficult to install a microphone immediately below an electrode inside an earpiece and use a signal of the microphone as a reference signal for noise removal.

The present disclosure has been made in view of such a situation, and an object thereof is to enable realization of acquisition of a cleaner biological signal.

### Solutions to Problems

A hearable device of the present disclosure is a hearable device including: a microphone; an inertial sensor; a biological sensor that measures a biological signal of a user; and a noise reduction unit that executes noise reduction of the biological signal using an output signal from at least one of the microphone or the inertial sensor as a reference signal.

An integrated circuit of the present disclosure is an integrated circuit including a noise reduction unit that executes noise reduction of a biological signal of a user measured by a biological sensor using an output signal from at least one of a microphone or an inertial sensor provided in a hearable device as a reference signal.

A biological signal measurement system of the present disclosure is a biological signal measurement system including: a hearable device; a noise reduction unit that executes noise reduction of a biological signal of a user measured by a biological sensor using an output signal from at least one of a microphone or an inertial sensor provided in the hearable device as a reference signal; a feature amount extraction unit that extracts a feature amount from the biological signal subjected to the noise reduction; and a user state estimation unit that estimates a state of the user on the basis of the extracted feature amount.

In the present disclosure, the noise reduction of the biological signal of the user measured by the biological sensor is executed using the output signal, as the reference signal, from at least one of the microphone or the inertial sensor provided in the hearable device.

### BRIEF DESCRIPTION OF DRAWINGS

FIG. 1 is a block diagram illustrating a configuration example of a biological signal measurement system of the present disclosure.

FIG. 2 is a flowchart for describing a flow of service providing processing.

FIG. 3 is a block diagram illustrating a configuration example of a TWS earphone.

FIG. 4 is a flowchart for describing an operation of the TWS earphone.

FIG. 5 is a diagram illustrating an appearance configuration example of a microphone.

FIG. 6 is a diagram illustrating arrangement examples of the microphone.

FIG. 7 is a diagram illustrating an arrangement example of the microphone.

### MODE FOR CARRYING OUT THE INVENTION

Hereinafter, a mode for carrying out the present disclosure (hereinafter referred to as an embodiment) will be described. Note that the description will be given in the following order.
1. Conventional technology and problems thereof
2. Biological signal measurement system and service providing processing of present disclosure
3. Configuration and operation of TWS earphone
4. Appearance and arrangement example of microphone
5. Others

### 1. Conventional Technology and Problems Thereof

In recent years, various services using a biological signal measurement technology around the ear by an electroencephalography (EEG) sensor, a photoplethysmography (PPG) sensor, or the like incorporated in a hearable device such as an earphone or a headphone have been proposed in many cases.

However, in a use condition actually assumed for the hearable device, there is a possibility that more noise is mixed in a body motion of a user and an external environment. That is, there is currently a problem in acquiring clean data at all times in daily life and providing a service using the data.

In addition, in a device that is required to be small and lightweight such as a true wireless stereo (TWS) earphone, it is required to effectively utilize resources particularly in a limited space and power consumption. In a hearable device having a good wearing feeling, if it becomes possible to measure a biological signal with low noise by low power consumption, the user can receive a high-quality service more easily.

Non-Patent Document 1 proposes a technology for removing noise using a signal of a microphone for an intra-aural electroencephalogram, but the microphone is built in a sponge-like earpiece provided with an electrode, and it is difficult to apply such a technology to a device for music listening.

If output signals of an acceleration sensor and a microphone built in many earphones can be made multimodal and used as a reference signal, noise generated by various operations in daily life can be efficiently removed, and only a clean biological signal can be acquired. In addition, while resources such as space and power consumption are limited, it is desirable that information obtained by an already implemented function can be used to the maximum. However, since the microphone built in the earphone is intended to acquire voice in the audible range, sensitivity to high frequency is relatively high, but sensitivity to low-frequency noise such as body motion is low, and thus the microphone is unlikely to serve as a reference signal for noise removal with respect to the biological signal as it is.

### 2. Biological Signal Measurement System and Service Providing Processing of Present Disclosure

In a biological signal measurement system of the present disclosure, information obtained by a function implemented in another application can be used to the maximum in a hearable device such as an earphone or a headphone in which resources such as space and power consumption are limited. As a result, noise mixed in a biological signal is efficiently removed by various operations in daily life, only a clean biological signal is acquired, and a service using the biological signal is provided to the user.

### Configuration of Biological Signal Measurement System

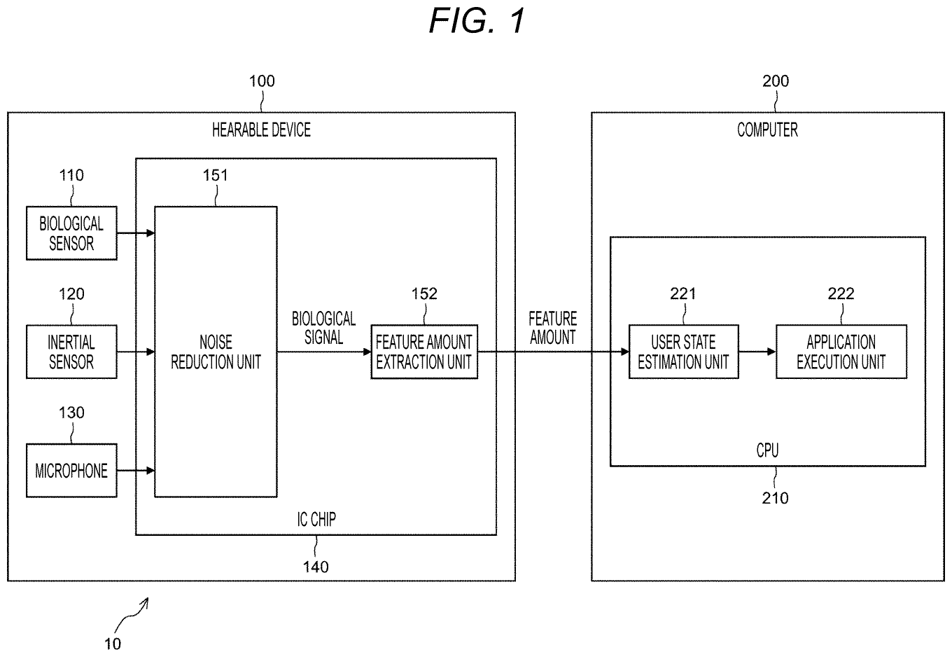

FIG. 1 is a block diagram illustrating a configuration example of the biological signal measurement system of the present disclosure.

A biological signal measurement system 10 illustrated in FIG. 1 includes a hearable device 100 and a computer 200.

The hearable device 100 is configured as an earphone, a headphone, or the like worn on the user's ear or around the ear. In a case where the hearable device 100 is configured as an earphone, the hearable device 100 may be a canal type earphone or an inner-ear type earphone. In addition, the hearable device 100 may be a TWS earphone.

Meanwhile, the computer 200 is configured as a smartphone, a tablet terminal, a personal computer (PC), or the like owned by the user. The computer 200 may be configured as a server on a cloud or the like.

The hearable device 100 can transmit various types of information to the computer 200 by wireless transmission via a wireless communication network such as Bluetooth low energy (BLE) (registered trademark) or a body area network (BAN).

The hearable device 100 includes a biological sensor 110, an inertial sensor 120, a microphone 130, and an integrated circuit (IC) chip 140. Note that, although not illustrated, the hearable device 100 also includes an acoustic function of outputting music and voice to the user's ear. Furthermore, the hearable device 100 may include other sensors such as an impedance sensor in addition to the inertial sensor 120 and the microphone 130.

The biological sensor 110 is provided inside a housing constituting an exterior of the hearable device 100 or provided in an earpiece or an earpad in a case where the hearable device 100 is configured as a canal type earphone. The biological sensor 110 measures a biological signal of the user wearing the hearable device 100 via an electrode in contact with the skin of the user. For example, the biological sensor 110 is configured as an EEG sensor, a PPG sensor, or the like, and measures an EEG signal or a PPG signal of the user as a biological signal.

The inertial sensor 120 is provided inside the housing of the hearable device 100. The inertial sensor 120 is configured as an acceleration sensor or a gyro sensor for tracking a user's behavior, and outputs, to the IC chip 140, a measurement signal obtained by measuring a six-axis acceleration or an angular velocity, as an output signal.

The microphone 130 is provided inside the housing of the hearable device 100. In a case where the hearable device 100 is configured as a canal type earphone, the microphone 130 may be provided in the earpiece or the earpad. The microphone 130 may be configured as, for example, a feedback microphone (FB microphone) for noise canceling. The microphone 130 outputs, to the IC chip 140, a sound collection signal obtained by collecting external sound of the hearable device 100, as an output signal. The low-frequency characteristic of the microphone 130 is set to at least −15 dB/Pa or more in order to improve sensitivity to low-frequency noise.

The IC chip 140 is configured as an integrated circuit according to the present disclosure, and realizes a function of efficiently removing noise mixed in a biological signal and acquiring only a clean biological signal. The IC chip 140 realizes a noise reduction unit 151 and a feature amount extraction unit 152 as functional blocks.

The noise reduction unit 151 executes noise reduction of the biological signal measured by the biological sensor 110 using the output signal from at least one of the inertial sensor 120 or the microphone 130 as a reference signal for noise removal. The biological signal subjected to the noise reduction is supplied to the feature amount extraction unit 152. In addition, in the noise reduction, an output signal from another sensor described above may be used as a reference signal for noise removal, as necessary.

The feature amount extraction unit 152 extracts various feature amounts from the biological signal subjected to the noise reduction performed by the noise reduction unit 151. The extracted feature amounts are transmitted to the computer 200 by wireless transmission and used for user state estimation.

The computer 200 includes a central processing unit (CPU) 210.

The CPU 210 implements various programs (algorithms) and realizes various functions by executing these programs (algorithms).

The CPU 210 realizes a user state estimation unit 221 and an application execution unit 222 as functional blocks.

The user state estimation unit 221 estimates the state of the user wearing the hearable device 100 on the basis of various feature amounts transmitted from the hearable device 100. The user state estimation performed by the user state estimation unit 221 includes user's emotion estimation, user's stress estimation, and the like.

The application execution unit 222 provides various services on the basis of the estimation result by the user state estimation unit 221.

### Flow of Service Providing Processing

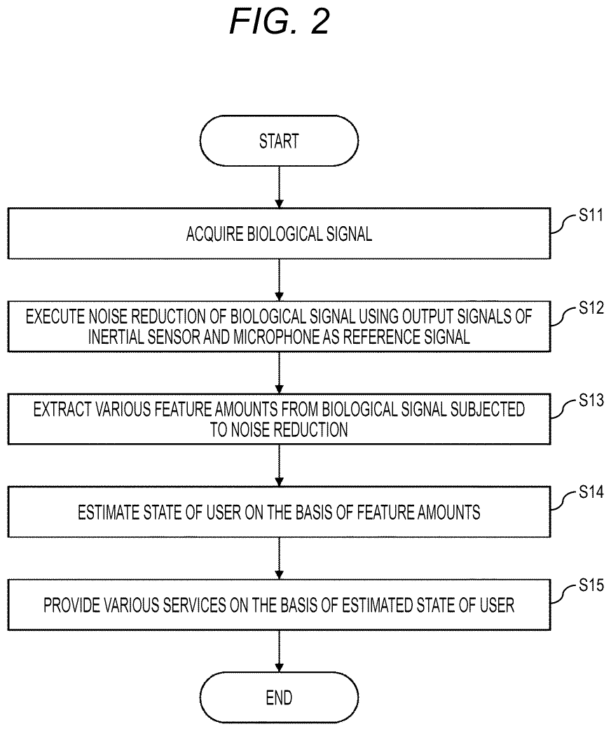

A flow of service providing processing performed by the biological signal measurement system 10 will be described with reference to a flowchart of FIG. 2.

In step S11, the noise reduction unit 151 of the hearable device 100 acquires a biological signal measured by the biological sensor 110.

In step S12, the noise reduction unit 151 executes noise reduction of the biological signal using output signals of the inertial sensor 120 and the microphone 130 as reference signals. Specifically, by using the output signal of the inertial sensor 120 having high sensitivity to low-frequency noise as a reference signal, low-frequency noise mixed in the biological signal can be removed. In addition, by using the output signal of the microphone 130 having high sensitivity to high-frequency noise as a reference signal, high-frequency noise mixed in the biological signal can be removed.

In step S13, the feature amount extraction unit 152 of the hearable device 100 extracts various feature amounts from the biological signal subjected to the noise reduction (from which the noise has been removed). The extracted various feature amounts are transmitted to the computer 200 by wireless transmission.

In step S14, the user state estimation unit 221 of the computer 200 estimates the state of the user on the basis of the various feature amounts transmitted from the hearable device 100.

In step S15, the application execution unit 222 of the computer 200 provides various services on the basis of the estimated state of the user.

According to the processing described above, by using the output signals from the inertial sensor 120 and the microphone 130 as reference signals, noise mixed in the biological signal by various operations in daily life can be efficiently removed. As a result, it is possible to realize acquisition of a cleaner biological signal, and eventually, it is possible to provide a high-quality service.

### 3. Configuration and Operation of TWS Earphone

Here, a configuration and an operation of the TWS earphone, which is an embodiment of the hearable device 100 of the present disclosure, will be described.

### Configuration of TWS Earphone

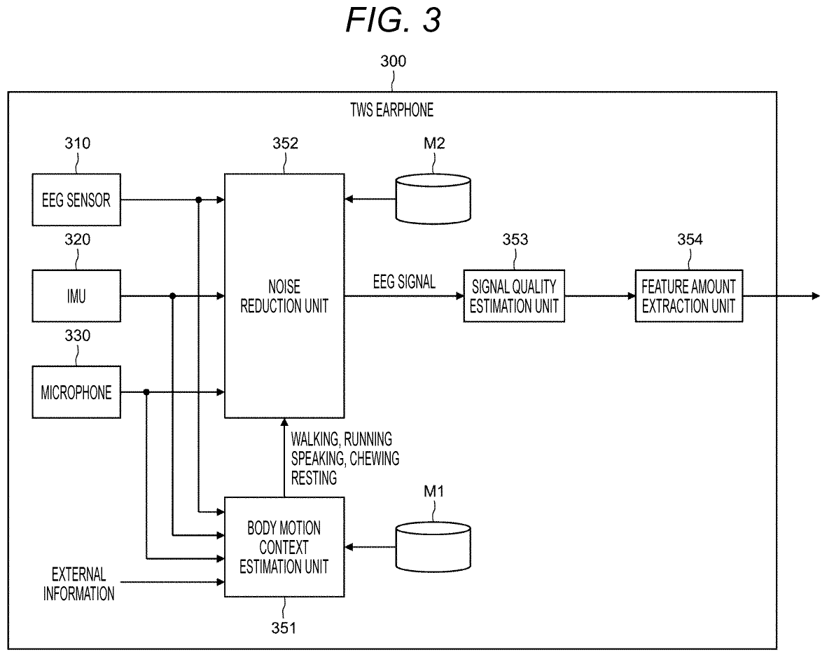

FIG. 3 is a block diagram illustrating a configuration example of the TWS earphone.

A TWS earphone 300 illustrated in FIG. 3 includes an EEG sensor 310, an IMU 320, a microphone 330, a body motion context estimation unit 351, a noise reduction unit 352, a signal quality estimation unit 353, and a feature amount extraction unit 354.

The EEG sensor 310 corresponds to the biological sensor 110 in FIG. 1, and measures an EEG signal in the ear of the user via, for example, an earpiece-type electrode.

The inertial measurement unit (IMU) 320 corresponds to the inertial sensor 120 in FIG. 1, and outputs, to the body motion context estimation unit 351 and the noise reduction unit 352, an acceleration signal obtained by measuring an inertial motion of the user, as an output signal. The IMU 320 may be a sensor implemented for tracking the user's behavior, or may be a sensor having a desired characteristic provided at a desired position for noise reduction separately from these sensors.

The microphone 330 corresponds to the microphone 130 in FIG. 1, and outputs, to the body motion context estimation unit 351 and the noise reduction unit 352, a sound collection signal obtained by collecting external sound of the TWS earphone 300, as an output signal. The microphone 330 may be an FB microphone implemented for noise canceling, or may be a microphone having a desired characteristic provided at a desired position for noise reduction separately from this sensor. It is desirable that the microphone 330 have excellent sensitivity in a low frequency range (in particular, a frequency band (about 1 to 40 Hz) related to the biological signal (EEG signal) of interest).

The body motion context estimation unit 351, the noise reduction unit 352, the signal quality estimation unit 353, and the feature amount extraction unit 354 can be realized by the IC chip 140 in FIG. 1.

The body motion context estimation unit 351 estimates a body motion context that classifies the type of the motion of the user's body by using a learned model M1 on the basis of the EEG signal from the EEG sensor 310 and the output signals from the IMU 320 and the microphone 330. Examples of the body motion context include walking, running, speaking, chewing, resting, and the like. For the estimation of the body motion context, external information obtained by another function of the TWS earphone 300, a smartphone (computer 200) as a communication partner, or the like may be used.

The noise reduction unit 352 executes noise reduction according to the body motion context estimated by the body motion context estimation unit 351 by using a learned model M2. The EEG signal subjected to the noise reduction is supplied to the signal quality estimation unit 353.

The signal quality estimation unit 353 estimates the signal quality of the EEG signal subjected to noise reduction, gives a label indicating the estimated signal quality to the EEG signal, and supplies the EEG signal to the feature amount extraction unit 354.

The feature amount extraction unit 354 extracts a feature amount from the EEG signal supplied from the signal quality estimation unit 353, and gives reliability based on the estimated signal quality (label) to the extracted feature amount. The feature amount to which the reliability is given is transmitted to the computer 200 (FIG. 1) by wireless transmission and used for user state estimation.

### Operation of TWS Earphone

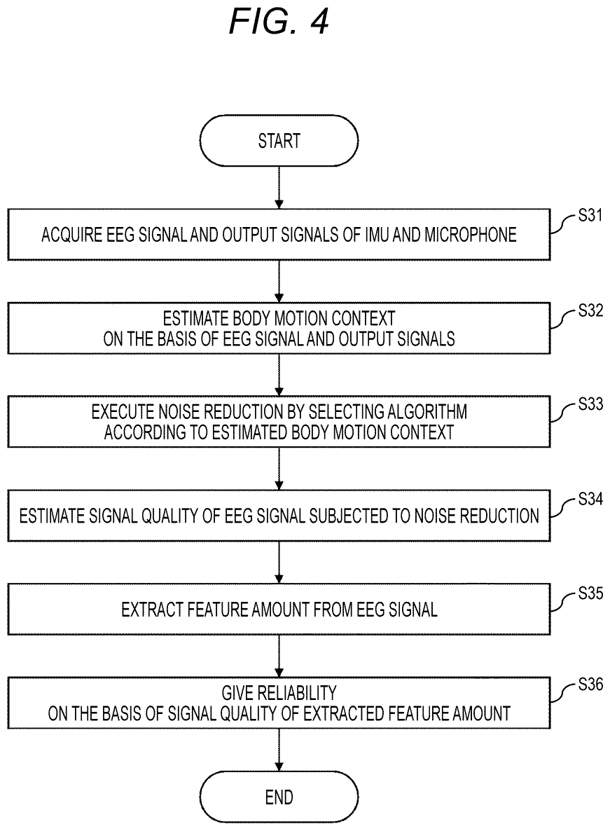

The operation of the TWS earphone 300 will be described with reference to a flowchart of FIG. 4.

In step S31, the body motion context estimation unit 351 and the noise reduction unit 352 acquire the EEG signal measured by the EEG sensor 310 and the output signals of the IMU 320 and the microphone 330. It is assumed that the output signals of the IMU 320 and the microphone 330 are time-synchronized with the EEG signal. The sampling rate of the output signals of the IMU 320 and the microphone 330 is equal to or higher than that of the EEG signal (preferably, 64 Hz or higher).

In step S32, the body motion context estimation unit 351 estimates the body motion context of the user on the basis of the EEG signal and the output signals. For example, the body motion of the user is classified into body motion contexts such as walking, running, speaking, chewing, and resting on the basis of the myoelectric noise mixed in the EEG signal, the motion of the trunk recognized from the inertial motion measured by the IMU 320, and the information of the vibration and voice of the housing input to the microphone 330. The body motion of the user may be, as necessary, classified into other body motion contexts such as sleeping or the body motion of the user may be classified on the basis of the user's situation (for example, operating TWS earphone 300, speaking, or the like) obtained as the external information described above.

In step S33, the noise reduction unit 352 executes noise reduction by selecting an algorithm for executing the noise reduction according to the body motion context estimated by the body motion context estimation unit 351. At this time, the noise reduction unit 352 changes the type of the output signal serving as the reference signal or changes the weighting of the output signal serving as the reference signal according to the estimated body motion context.

For example, in a case of a large and gentle body motion such as walking or running, the noise reduction is executed by an adaptive filter using the output signal of the IMU 320. In addition, in a case of a body motion in which relatively high frequency noise is dominant, such as speaking or chewing, the noise reduction is executed, for example, by an adaptive filter using the output signal of the microphone 330 or by multivariate empirical mode decomposition (EMD) analysis.

Note that the type of algorithm selected according to the body motion context is not limited to that described above, and a deep neural network (DNN) model according to the body motion context may be selected and applied. Furthermore, the noise reduction can be executed by one DNN model including estimation of the body motion context. Note that these noise removal algorithms may be periodically updated at the time of firmware update or the like, or may be automatically updated by performing parameter adjustment or the like by the user.

In order to efficiently execute signal processing by these algorithms, the noise reduction unit 352 may switch a processor (IC) that executes noise reduction according to the selected algorithm. For example, a control IC is used for the numerical analysis algorithm, and an artificial intelligence (AI) IC is used for the DNN model. More specifically, the AI IC may be used for the noise reduction algorithm, and the control IC may be used for feature amount extraction in the subsequent stage. In addition, in the noise reduction algorithm, the AI IC can be used for an algorithm based on the DNN model or the like, and the control IC can be used for an algorithm based on the adaptive filter, multivariate EMD analysis, or the like. As described above, by adaptively selecting the IC according to the type of calculation, it is possible to achieve high-speed calculation and low power consumption.

In addition, the noise reduction unit 352 may execute the noise reduction using an impedance measurement value of an electrode of the biological sensor (EEG sensor 310) as a reference signal in addition to the output signals of the IMU 320 and the microphone 330.

After the noise reduction is executed in this manner, in step S34, the signal quality estimation unit 353 estimates the signal quality of the EEG signal subjected to the noise reduction, thereby giving a label indicating the signal quality to the EEG signal.

In step S35, the feature amount extraction unit 354 extracts a feature amount from the labeled EEG signal. The extracted feature amount is given reliability based on the label given to the EEG signal, and is transmitted to the computer 200 by wireless transmission.

As described above, the wireless transmission is performed after the extraction of the feature amount, so that the amount of information transmitted to the computer 200 can be reduced in the wireless transmission in which the transmission band and the power consumption are limited. Furthermore, by giving the reliability to the feature amount, the accuracy of the user's emotion estimation and stress estimation in the computer 200 can be improved.

In the computer 200, the user state estimation unit 221 estimates the emotion of the user by the emotion estimation algorithm and estimates the mental fatigue level of the user by the stress estimation algorithm on the basis of the feature amount transmitted from the TWS earphone 300 and the reliability thereof. The application execution unit 222 processes the estimation result of the user state estimation unit 221 and presents the estimation result to the user by an image, voice, or the like via a user interface (UI).

According to the processing described above, since the noise reduction according to the body motion context of the user is executed, the noise mixed in the biological signal by various operations in daily life can be efficiently removed, and as a result, it is possible to realize acquisition of a cleaner biological signal.

In the above description, in view of limitations on the transmission band and power consumption in the wireless transmission, the wireless transmission is performed after the extraction of the feature amount. Not limited to this, in a case where the processing capability of the IC chip on an edge terminal (TWS earphone 300) side or random access memory (RAM) capacity is improved in the future, the processing after the extraction of the feature amount to the user state estimation may be performed on the edge terminal side. In addition, in a case where the wireless transmission technology is improved, all of the EEG signal and the output signal (reference signal) may be transmitted from the edge terminal to the CPU (computer 200) side, and the estimation of the body motion context and the processing after the noise reduction may be executed on the CPU side.

### 4. Appearance and Arrangement Example of Microphone

Here, an appearance and an arrangement example of the microphone 330 included in the TWS earphone 300 will be described.

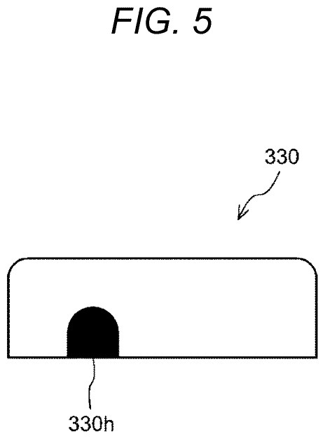

FIG. 5 is a diagram illustrating an appearance configuration example of the microphone 330.

The microphone 330 is provided with an IC chip or the like in a case formed in a flat rectangular parallelepiped shape, for example, and a sound collection hole 330h is formed in a part of the case. The microphone 330 may be a micro electronics mechanical system (MEMS) microphone or may be an electrolytic capacitor microphone. In a case where the microphone 330 is configured as an FB microphone for noise canceling, noise canceling based on external sound collected from the sound collection hole 330h is performed.

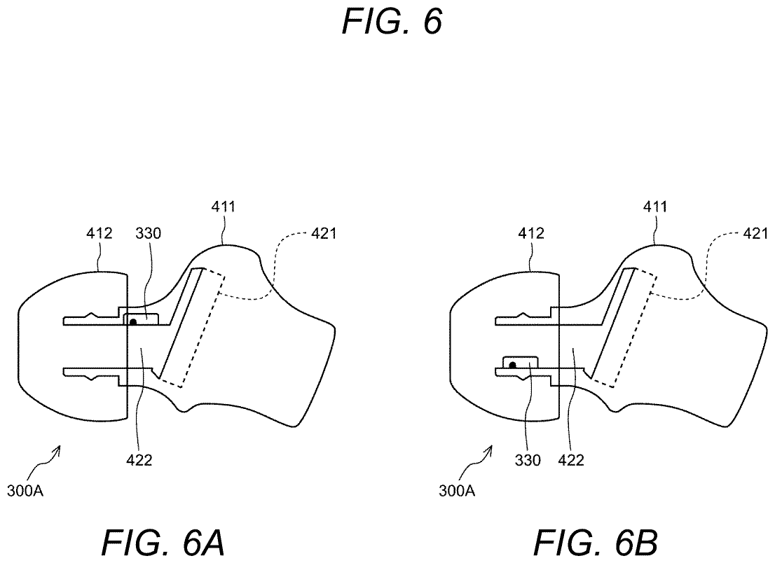

FIG. 6 is a diagram illustrating arrangement examples of the microphone 330 in a case where the TWS earphone 300 is a canal type earphone.

Earphones 300A illustrated in A and B of FIG. 6 each include a housing 411 and an earpiece 412.

A driver unit 421 that converts a voice signal into sound is built in the housing 411. The sound emitted from the driver unit 421 is delivered to the user's ear through a sound conduit 422. The earpiece 412 is detachably attached to a tip portion of the sound conduit 422, and has a function of transmitting the sound emitted from the sound conduit 422 to the ear without distortion while sealing the ear of the user and blocking the surrounding sound. In the earphone 300A of the present embodiment, the earpiece 412 also functions as an electrode of the EEG sensor 310.

In the canal type earphone 300A configured as described above, as illustrated in A of FIG. 6, the microphone 330 may be arranged inside the housing 411 and configured as an FB microphone for noise canceling that collects sound in the sound conduit 422. This arrangement is an arrangement based on acoustic characteristics of noise canceling. In this case, cost and power consumption can be suppressed as compared with a configuration in which a microphone is provided separately from the FB microphone for noise canceling.

In addition, as illustrated in B of FIG. 6, the microphone 330 may be arranged inside the sound conduit 422 and in the immediate vicinity of the earpiece 412. This arrangement is an arrangement specialized for removing noise included in the EEG signal from the EEG sensor 310.

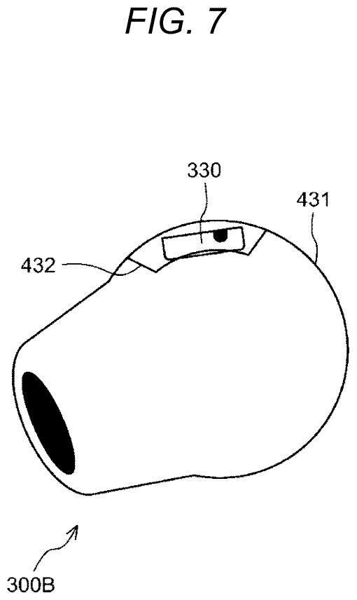

FIG. 7 is a diagram illustrating an arrangement example of the microphone 330 in a case where the TWS earphone 300 is an inner-ear type earphone.

An earphone 300B illustrated in FIG. 7 includes a housing 431 and an electrode 432.

The electrode 432 is provided at a portion in contact with the concha at the entrance of the user's ear in the housing 431, and functions as an electrode of the EEG sensor 310. The electrode 432 may include a conductive resin or may include metal.

In the inner-ear type earphone 300B configured as described above, the microphone 330 requires to be arranged inside the housing 431 and in the immediate vicinity of the electrode 432.

This arrangement is an arrangement specialized for removing noise included in the EEG signal from the EEG sensor 310.

### 5. Others

The embodiment described above realizes acquisition of the EEG signal by the TWS earphone and provision of a service based thereon. The hardware configuration to which the technology according to the present disclosure can be applied is not limited to earphones, and may be headband type headphones, a head mounted display (HMD), or the like. In addition, the biological signal handled in the technology according to the present disclosure is not limited to the EEG signal, and may be a biological signal measured by a photoelectric pulse wave sensor, a myoelectric sensor, an electro-oculography sensor, or the like.

Note that the effects described in the present specification are merely examples and are not limited, and other effects may be provided.

Furthermore, the embodiment to which the technology according to the present disclosure is applied is not limited to the embodiment described above, and various modifications can be made without departing from the scope of the technology according to the present disclosure.

Moreover, the present disclosure may have the following configurations.
(1)

A hearable device including:
a microphone;
an inertial sensor;
a biological sensor that measures a biological signal of a user; and
a noise reduction unit that executes noise reduction of the biological signal using an output signal from at least one of the microphone or the inertial sensor as a reference signal.
(2)

The hearable device according to (1), in which
the microphone is provided inside a housing.
(3)

The hearable device according to (1) or (2), in which
a low-frequency characteristic of the microphone is at least −15 dB/Pa or more.
(4)

The hearable device according to any one of (1) to (3), further including
a feature amount extraction unit that extracts a feature amount from the biological signal subjected to the noise reduction, in which
the feature amount is used for state estimation of the user.
(5)

The hearable device according to (4), in which
the feature amount is transmitted to a computer that provides a service based on the estimated state of the user.
(6)

The hearable device according to (4) or (5), in which
the state estimation of the user includes emotion estimation of the user or stress estimation of the user.
(7)

The hearable device according to any one of (4) to (6), further including:
a signal quality estimation unit that estimates signal quality of the biological signal subjected to the noise reduction, in which
the feature amount extraction unit gives reliability based on the estimated signal quality to the feature amount extracted from the biological signal.
(8)

The hearable device according to any one of (1) to (7), further including
a body motion context estimation unit that estimates a body motion context of the user on the basis of the biological signal and the output signal, in which
the noise reduction unit executes the noise reduction according to the estimated body motion context.
(9)

The hearable device according to (8), in which
the noise reduction unit selects an algorithm for executing the noise reduction according to the estimated body motion context.
(10)

The hearable device according to (9), in which
the noise reduction unit switches a processor that executes the noise reduction according to the selected algorithm.
(11)

The hearable device according to any one of (8) to (10), in which
the noise reduction unit changes a type or weighting of the output signal used as the reference signal according to the estimated body motion context.
(12)

The hearable device according to any one of (1) to (11), in which
the microphone is provided in an immediate vicinity of an electrode of the biological sensor.
(13)

The hearable device according to any one of (1) to (12), in which
the microphone is a feedback microphone for noise canceling.
(14)

The hearable device according to any one of (1) to (13), in which
the noise reduction unit executes the noise reduction using an impedance measurement value of an electrode of the biological sensor as the reference signal in addition to the output signal.
(15)

The hearable device according to any one of (1) to (14), in which
the biological sensor includes at least one of an electroencephalography (EEG) sensor or a photoplethysmography (PPG) sensor.
(16)

The hearable device according to any one of (1) to (15), in which
the inertial sensor includes at least one of an acceleration sensor or a gyro sensor.
(17)

The hearable device according to any one of (1) to (16), in which
the microphone is a micro electronics mechanical system (MEMS) microphone or an electrolytic capacitor microphone.
(18)

The hearable device according to any one of (1) to (17), in which
the hearable device is configured as an earphone or a headphone.
(19)

An integrated circuit including
a noise reduction unit that executes noise reduction of a biological signal of a user measured by a biological sensor using an output signal from at least one of a microphone or an inertial sensor provided in a hearable device as a reference signal.
(20)

A biological signal measurement system including:
a hearable device;
a noise reduction unit that executes noise reduction of a biological signal of a user measured by a biological sensor using an output signal from at least one of a microphone or an inertial sensor provided in the hearable device as a reference signal;
a feature amount extraction unit that extracts a feature amount from the biological signal subjected to the noise reduction; and
a user state estimation unit that estimates a state of the user on the basis of the extracted feature amount.

### REFERENCE SIGNS LIST

10 Biological signal measurement system
100 Hearable device
110 Biological sensor
120 Inertial sensor
130 Microphone
140 IC chip
151 Noise reduction unit
152 Feature amount extraction unit
200 Computer
210 CPU
221 User state estimation unit
222 Application execution unit
300 TWS earphone
310 EEG sensor
320 IMU
330 Microphone
351 Body motion context estimation unit
352 Noise reduction unit
353 Signal quality estimation unit
354 Feature amount extraction unit

What is claimed is:

1. A hearable device comprising:
a microphone;
an inertial sensor;
a biological sensor that measures a biological signal of a user; and
a noise reduction unit that executes noise reduction of the biological signal using an output signal from at least one of the microphone or the inertial sensor as a reference signal.

2. The hearable device according to claim 1, wherein
the microphone is provided inside a housing.

3. The hearable device according to claim 1, wherein
a low-frequency characteristic of the microphone is at least −15 dB/Pa or more.

4. The hearable device according to claim 1, further comprising
a feature amount extraction unit that extracts a feature amount from the biological signal subjected to the noise reduction, wherein
the feature amount is used for state estimation of the user.

5. The hearable device according to claim 4, wherein
the feature amount is transmitted to a computer that provides a service based on the estimated state of the user.

6. The hearable device according to claim 4, wherein
the state estimation of the user includes emotion estimation of the user or stress estimation of the user.

7. The hearable device according to claim 4, further comprising
a signal quality estimation unit that estimates signal quality of the biological signal subjected to the noise reduction, wherein
the feature amount extraction unit gives reliability based on the estimated signal quality to the feature amount extracted from the biological signal.

8. The hearable device according to claim 1, further comprising
a body motion context estimation unit that estimates a body motion context of the user on a basis of the biological signal and the output signal, wherein
the noise reduction unit executes the noise reduction according to the estimated body motion context.

9. The hearable device according to claim 8, wherein
the noise reduction unit selects an algorithm for executing the noise reduction according to the estimated body motion context.

10. The hearable device according to claim 9, wherein
the noise reduction unit switches a processor that executes the noise reduction according to the selected algorithm.

11. The hearable device according to claim 8, wherein
the noise reduction unit changes a type or weighting of the output signal used as the reference signal according to the estimated body motion context.

12. The hearable device according to claim 1, wherein
the microphone is provided in an immediate vicinity of an electrode of the biological sensor.

13. The hearable device according to claim 1, wherein
the microphone is a feedback microphone for noise canceling.

14. The hearable device according to claim 1, wherein
the noise reduction unit executes the noise reduction using an impedance measurement value of an electrode of the biological sensor as the reference signal in addition to the output signal.

15. The hearable device according to claim 1, wherein
the biological sensor includes at least one of an electroencephalography (EEG) sensor or a photoplethysmography (PPG) sensor.

16. The hearable device according to claim 1, wherein
the inertial sensor includes at least one of an acceleration sensor or a gyro sensor.

17. The hearable device according to claim 1, wherein
the microphone is a micro electronics mechanical system (MEMS) microphone or an electrolytic capacitor microphone.

18. The hearable device according to claim 1, wherein
the hearable device is configured as an earphone or a headphone.

19. An integrated circuit comprising
a noise reduction unit that executes noise reduction of a biological signal of a user measured by a biological sensor using an output signal from at least one of a microphone or an inertial Sensor provided in a hearable device as a reference signal.

20. A biological signal measurement system comprising:
a hearable device;
a noise reduction unit that executes noise reduction of a biological signal of a user measured by a biological sensor using an output signal from at least one of a microphone or an inertial sensor provided in the hearable device as a reference signal;
a feature amount extraction unit that extracts a feature amount from the biological signal subjected to the noise reduction; and
a user state estimation unit that estimates a state of the user on a basis of the extracted feature amount.
